(19) United States
(12) Patent Application Publication
(10) Pub. No.: US 2011/0275929 A1
(43) Pub. Date: Nov. 10, 2011

(54) Device and method for measuring contrast agent

(75) Inventors: Valerij Kiselev, Freiburg (DE); Peter Gall, Freiburg (DE); Elias Kellner, Schwaigern (DE)

(73) Assignee: Universitaetsklinikum Freiburg, Freiburg (DE)

(21) Appl. No.: 13/067,040

(22) Filed: May 4, 2011

(30) Foreign Application Priority Data
May 7, 2010 (DE) 10 2010 028 749.0

(51) Int. Cl. A61B 5/055

(52) U.S. Cl. 600/419

(57) ABSTRACT

A tomography device (3) producing magnetic resonance (MR) images of a part of the body of a living organism (1) disposed in a measurement volume of the tomography device (3), with a first measurement unit (4a) for acquiring a spatially resolved temporal series of MR images of the part of the body under examination, wherein the temporal series of MR images represents the passage of a contrast agent injected into the blood stream of the living organism through an organ located in the part of the body under examination, is characterized in that at least one further measurement unit (4b) is provided that comprises a local receiver coil that measures, close to at least one artery that supplies the part of the body (2) of the living organism disposed in the tomography device, the concentration of contrast agent with temporal resolution and concurrently with measurement of the temporal series of MR images determined by the first measurement unit. A system and an associated method are thereby provided that permit simultaneous measurement of large vessels and tissue with one and the same sequence with an adapted dynamic range.

This application claims Paris Convention priority of DE 10 2010 028 749.0 filed May 7, 2010 the complete disclosure of which is hereby incorporated by reference.

### BACKGROUND OF THE INVENTION

The invention relates to a tomography device for producing magnetic resonance (MR) images of a part of the body of a living organism disposed in a measurement volume of the tomography device, with a first measurement unit for acquiring a spatially resolved temporal series of MR images of the part of the body under examination, wherein the temporal series of MR images represents the passage of a contrast agent injected into the blood stream of the living organism through an organ located in the part of the body under examination.

Such a device is known from EP 0 958 503 B1.

The aim of dynamic susceptibility-compensated (DSC) measurement is to determine perfusion parameters such as cerebral blood volume (CBV), cerebral blood flow (CBF), and mean transit time (MTT) with spatial resolution. If a contrast agent bolus is injected into a vein of the arm, the passage of this bolus through the brain produces a time-variable contrast in the magnetic resonance (MR) image. Fast MR sequences (e.g. echo planar imaging (EPI)) permit the passage of this bolus to be measured with temporal and spatial resolution. From a temporal series measured in this way, it is possible to determine the change in the relaxation rate of each voxel. The contrast agent concentration can be determined approximately from the change in the relaxation rate.

The parameters stated above can be determined using the tracer kinetic model [1, 2, 3]. This model establishes a causal relationship between the progression of the contrast agent concentration in the arterial input (cᵢₙ (t)) and the progression of the contrast agent concentration in the voxel under consideration (cₜ (t)). The parameters stated above can be determined by comparing the curves of the two progressions.

The dynamic range of the measurement is limited by the concentration of the contrast agent compared with the echo time (TE) of the MR sequence used. In the case of a short TE, it is possible to measure the change in the relaxation rate in large vessels, whereas in tissue in which the CBV is very small, the effect of the contrast agent is no longer visible. In the case of a long TE, the effect in the tissue is clearly visible and the magnetization in large vessels is then almost completely relaxed and does not therefore produce a signal. A very short echo time would permit measurement of the arterial blood but it is subject to a lower limit imposed by the requirements of the imaging so that the magnetization is greatly relaxed in arteries. In a standard protocol, TE tissue is optimized. There are methods by which multiple echoes of the same excitation can be acquired [4], but it has been shown that the shortest echo time is still too long for measurement of the arterial signal.

Moreover, the presence of contrast agent results in a considerable displacement of the Larmor frequency. This disturbs the spatial encoding in fast MR sequences, such as the EPI sequence. This is manifested by an apparent movement of a large vessel through the image. The direction of the movement depends on the encoding scheme used.

The tracer kinetic model requires that cᵢₙ be the direct input of the voxel under consideration. As explained above, the AIF can alternatively be determined further away in the vascular tree. Over this additional distance, the shape and the arrival time of the bolus changes because of the blood flow conditions. To minimize these effects, it has been suggested that an individual AIF be determined for a certain region of the brain instead of a global AIF [5]. However, with this method, local AIFs are determined in arteries that are very small by comparison with the voxel size because of the relatively low spatial resolution. This effect, called the partial volume effect, results in a serious loss of the arterial contribution in the measured signal.

To minimize partial volume effects, van Osch et al. in [6] have proposed recording the complex signal during the DSC measurement. If a vessel is chosen that is parallel to the magnetic field, the contribution of the large vessel can be separated from that of the surrounding tissue. However, such a vessel is hard to find. In particular, blood vessels that can be used as local AIFs are not usually parallel to the magnetic field and are thus not correctable. Large vessels such as the internal carotid artery are nearly parallel to the magnetic field, but the high contrast agent concentration per voxel exceeds the dynamic range of a typical measurement.

The currently most frequently deployed method of DSC evaluation is based on the selection of a global AIF. The selection can be made manually with the user selecting a voxel whose signal he considers to be a suitable AIF.

It has been shown that the resulting perfusion parameters depend strongly on the user. To obtain more comparable and reproducible results, approaches for automatic AIF selection were therefore proposed [7, 8, 9]. Just as in manual selection, an AIF distorted by partial volume effects and apparent motion is obtained.

Patent EP 0 958 503 B1 proposes measurement of the AIF in the neck of the patient. Because the signal is not acquired using a separate coil, the problem of the limited dynamic range remains.

A further problem in determining the perfusion parameters is the time resolution of the bolus passage. This is particularly insufficient for arteries where the blood flow is high.

The problems of the prior art stated above can be summarized as follows:
Greatly differing concentration of the contrast agent in large vessels and tissue make precise, simultaneous measurement of large vessels and tissue with the same sequence impossible.
This results in a decline in the arterial signal below the noise level and in a shift in the phase of the signal.
The time resolution of the DSC measurement is very low, especially for the AIF.
Arterial input functions that can be determined by means of the prior art techniques are distorted by partial volume effects.

The object of this invention is to provide a system and an associated method that permits simultaneous measurement of large vessels and tissue with one and the same sequence with an adapted dynamic range.

### SUMMARY OF THE INVENTION

This task is inventively solved in a manner that is both surprisingly simple and effective in that at least one further measurement unit is provided that comprises a local receiver coil that measures, close to at least one artery that supplies the part of the body of the living organism disposed in the tomography device, the concentration of contrast agent with temporal resolution and concurrently with measurement of the temporal series of MR images determined by the first measurement unit. According to the prior art, there is only one main measurement unit with which an attempt is made to measure vessels and tissue. In accordance with the invention, at least one further measurement unit is provided for adjacent disposition on at least one artery. This additional measurement unit essentially measures the contrast agent concentration on the at least one artery simultaneously with the temporal series determined by the tomography device.

One typical, but non-exclusive application of the invention is to examine the brain of a human patient under examination. In this case, the at least one artery would be the carotid arteries in the neck that supply blood to the brain.

Adjacent disposition means that the artery is in the sensitivity range of the further, additional measurement unit. Essentially simultaneously means that the time offset of the measurement with the further measurement unit is smaller than the time resolution of the temporal series measured by the tomography device. This measurement can therefore be made simultaneously or with a slight time offset.

The at least one further measurement unit can be disposed adjacently on one or more arteries. The further measurement units, however, can also be disposed at different locations along one artery.

One especially advantageous embodiment of the invention is characterized in that the further measurement unit is constituted as a surface coil. The measurement unit constituted as a surface coil can be attached especially simply to arteries lying near to the body surface of a mammal.

In a further embodiment, the further measurement unit is connected to a dedicated data acquisition channel of the tomography device. By using a dedicated acquisition channel, the measurement with the determination device is not impaired by the at least one further measurement unit. This enables the essentially simultaneous measurement.

An embodiment of the invention is advantageous in which the further measurement unit encloses a smaller volume than the first measurement unit. This makes the further measurement unit easier to handle and it can be flexibly attached even to difficult-to-access parts of the body.

One especially preferred embodiment of the invention has at least two further measurement units. By using at least two measurement units, whereby spatial differentiation of signal sources is possible in the living organisms under examination, the AIF can be determined separately for each artery. Moreover, the artery under consideration can be observed in a manner such that it is differentiated from the surrounding tissue.

The invention also concerns a method for operating an inventive tomography device that is characterized in that excitation of the magnetization of the blood and/or contrast agent carried in the at least one artery of the living organism under examination is performed chronologically before and/or after the imaging portion of the measurement sequence of the first measurement unit. Because the magnetization for measurement of the contrast agent concentration is excited at an instant at which the tomography device is not performing imaging, measurement with the tomography device is not impaired. The excitation required for measurement with the at least one further measurement unit is therefore performed at an instant at which radio-frequency-pulse-free and gradient-free time windows are available in the sequence used by the tomography device.

A further variant of the method is characterized in that a read-out operation of the further measurement unit is performed chronologically before and/or after the imaging portion of the measurement sequence of the first measurement unit.

One variant of the inventive method must be seen as especially advantageous in which the excitation of the magnetization of the blood and/or contrast agent carried in the artery of the living organism under examination is performed before and/or after the imaging portion of the measurement sequence of the first measurement unit, wherein the read-out operation of the further measurement unit is performed during and/or after the imaging portion of the measurement sequence of the first measurement unit. In this way, an especially long time is available for the measurement by the at least one measurement unit. Moreover, the measurement by the at least one measurement unit can be repeated after repeated excitation of the magnetization after the imaging portion of the measurement sequence of the determination device.

A further advantageous variant of the inventive method is characterized in that the excitation slice is chosen to be sufficiently thick that the change in the signal acquired by the further measurement unit caused by the time-variable blood flow in the at least one artery is negligibly small. In this way, it is no longer necessary to determine the time-variable blood flow in the at least one artery. This permits especially simple evaluation of the signal by the at least one measurement unit.

A variant of the inventive method is preferable in which at least two excitation slices are used. By exciting multiple slices, the blood flow in the at least one artery can be determined. The determined blood flow can be used for correction of the signals modulated by the blood flow by the at least one further measurement unit. This embodiment is especially advantageous if the anatomy of the living organism under examination does not permit a sufficiently thick excitation slice.

In a further variant of the inventive method, the contrast between the artery and the tissue surrounding the artery can be set using the relevant flip angle of the excitations of the magnetization. Adapting the flip angle chosen for excitation of the magnetization advantageously enables the contrast between the artery being observed and the surrounding tissue to be set to be as large as possible so that determination of the arterial contrast agent concentration by the at least one measurement unit is simple.

In another variant of the inventive method, subsequent to the excitation and read-out operation executed before the imaging portion of the measurement sequence, one or more additional excitations with a read-out operation are appended after the imaging portion of the measurement sequence and with variable starting times. In this way, the transverse tissue magnetization surrounding the artery is additionally suppressed. Moreover, the temporal sampling rate can be increased according to the number of read-out operations.

A variant of the inventive method is advantageous in which the contrast agent concentration is determined by isolating the portion of the spectrum caused by the contrast agent out of the measured signal. By determining the contrast agent concentration using the complex spectrum measured by the at least one further measurement unit, the concentration is especially stable and can be performed precisely by isolation of the corresponding resonance function.

Further advantages of the invention can be derived from the description and the drawing. Equally, according to the invention, the characteristics stated above and further explained below can be used singly or in any combination. The embodiments shown and described are not intended as an exhaustive list but are examples used to describe the invention.

The invention is depicted in the drawing and is explained in more detail using embodiments.

The figures show:

### BRIEF DESCRIPTION OF THE DRAWING

FIG. 1A schematic diagram of an inventive tomography device with a further measurement unit; and

FIG. 2 A sequence diagram for an inventive tomography device; upper line: all excitation pulses and read-out of the first measurement unit; center line: reaction of the excited slice to the excitation; lower line: read-out sequence of the further measurement unit; left half: first excited slice; right half: second excited slice.

### DESCRIPTION OF THE PREFERRED EMBODIMENT

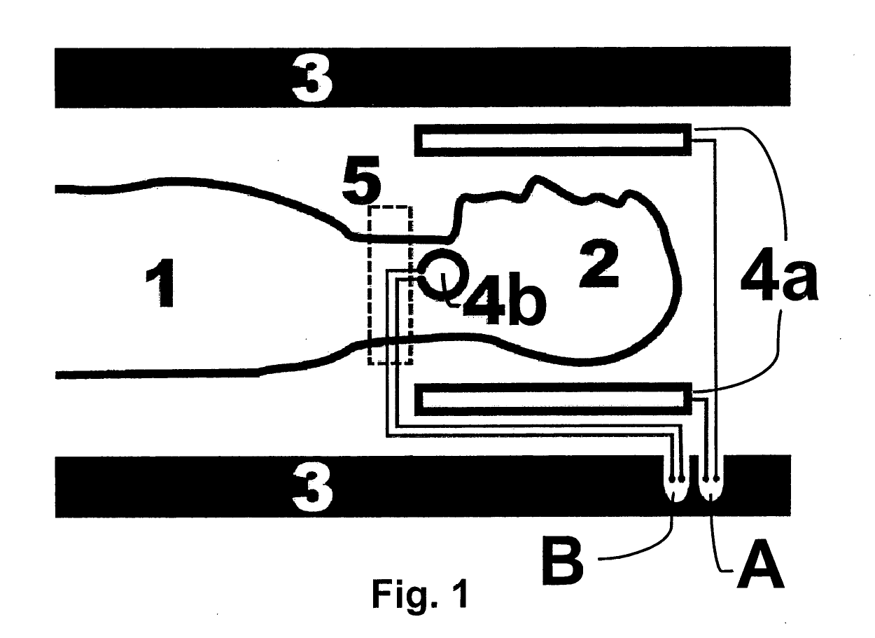

In the schematic representation FIG. 1, one part of the body 2 (in this case the head) of the living organism 1 under examination (in this case a human being) is positioned in the measurement volume defined by the first measurement unit 4a of the tomography device 3. The further measurement unit 4b constituted as a surface coil is attached to the neck of the patient. The magnetization from the excitation slice 5 is used as a signal source for the further measurement unit 4b.

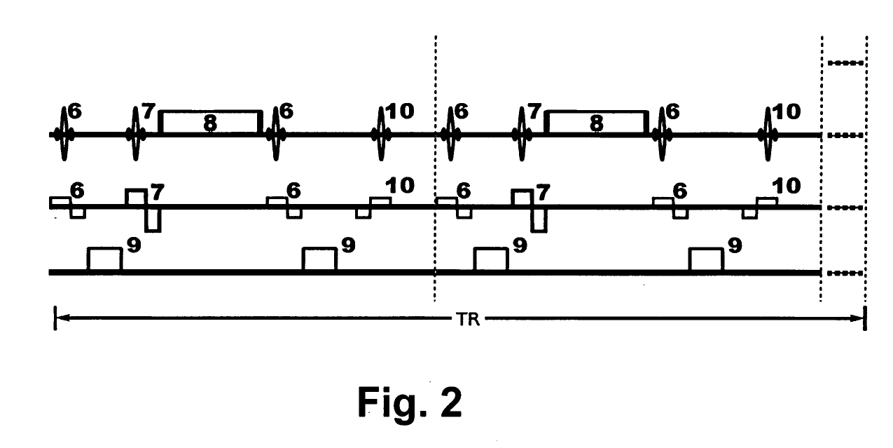

FIG. 2 shows the measurement sequence for operation of an inventive tomography device. In the sequence, after excitation of the tissue magnetization 7 in each slice, an imaging block 8 is inserted to perform the in-slice encoding. The excitation of the contrast agent magnetization 6 in the slice that results in the signal 9 in the further measurement unit 4b can be performed before and/or after the imaging block 8 and the excitation 7. In the case of excitation before the imaging block 8, the influence of the imaging gradients during the imaging block 8 during interpretation of the signal is taken into account by the further measurement unit 4b.

The signal of the further measurement unit 4b is recorded with the end of the excitation pulse 6 using at least one dedicated data acquisition channel B. This signal is temporally better sampled by the number of slices than the temporal series that is measured for each voxel.

The large and pulsatile flow, approximately 1 m/s in the systole and approximately 0.2 m/s in the diastole, results in a spatial offset and in smearing of the excited slice 5 required for the further measurement unit 4b. If the spatial offset is approximately the same size as the thickness of the excited slice 5, the signal measured by the further measurement unit 4b is significantly modulated by the flow. This modulation must be suppressed or determined in order to determine the concentration of the contrast agent.

Suppression succeeds if the excited slice 5 chosen is as thick as the measurement circumstances will permit. One typical suitable slice thickness is approximately 10 cm. With a flow rate of 1 m/s, the spatial offset of the fastest slice front is 5 cm, which is within the duration of the imaging block of 50 ms. Because the blending of excited magnetization is performed with non-excited magnetization at the edge of the slice 5, this effect can be minimized by choosing a thick slice.

The parameters of the modulation can be determined by exciting multiple thin slices at variable distances from the location of the further measurement unit 4b. The modulation caused by blood flow is encoded in this way. The encoding scheme then uses the flow modulation from the signals measured with the further measurement units.

Multiple further measurement units disposed at different locations on the neck of a human being can be used to achieve spatial differentiation of the carotid arteries of the neck that are close to the surface. The gradients of the imaging sequence can also be used for imaging with the inventive, at least one further measurement unit.

The choice of flip angle for the inventive additional excitations 6 and 10 (in addition to excitation 7 of prior art) must be performed in such a way that the contrast between the signal amplitude in the tissue and in the artery is as large as possible. The flip angle of the last excitation per slice block 10 must be chosen such that the tissue magnetization on which the excitation 6 acts is minimal at the instant of measurement of the next slice.

### LIST OF REFERENCE SYMBOLS

1 Living organism under examination
2 Part of the body under examination
3 Tomography device
4a First measurement unit (tissue)
4b Further measurement unit (contrast agent/blood)
5 Excited slice (for 4b)
6 Excitation pulse for the further measurement unit 4b
7 Excitation pulse for the first measurement unit 4a
8 Imaging block
9 Read-out of the signal of the further measurement unit
10 Additional excitation pulse
A Data channel first measurement unit
B Data channel further measurement unit

### REFERENCES

[1] L. Østergaard, R. Weisskoff, D. Chesler, C. Gyldensted, and B. Rosen: High resolution measurement of cerebral blood flow using intravascular tracer bolus passages. Part I: Mathematical approach and statistical analysis. Magn Reson Med, 36(5):715-25, 1996
[2] L. Østergaard, A. Sørensen, K. Kwong, R. Weisskoff, C Gyldensted, and B. Rosen: High resolution measurement of cerebral blood flow using intravascular tracer bolus passages. Part II: Experimental comparison and preliminary results. Magn Reson Med, 36(5):715-25, 1996
[3] L. Østergaard: Patent DE000060024073T2: System zu Bestimmung hämodynamischer Indizes mittels tomographischer Daten.
[4] R. Newbould, S. Skare, T. Jochimsen, M. Alley, M. Moseley, G. Albers, R. Bammer: Perfusion mapping with multiecho multishot parallel imaging EPI. Magn Reson Med, 58(1):70-81, 2007
[5] F. Calamante, M. Morup, L. Hansen: Defining a local arterial input function for perfusion MRI using independent component analysis. Magn Reson Med, 52(4):789-797, 2004
[6] M. van Osch, E. Vonken, M. Viergever, J. van der Grond, and C. Bakker: Measuring the arterial input function with gradient echo sequences. Magn Reson Med., 49:1067-76, 2003
[7] T. J. Carroll, H. A. Rowley, and V. M. Haughton: Automatic calculation of the arterial input function for cerebral perfusion imaging with mr imaging. Radiology, 227(2):593-600, May 2003
[8] T. J. Carroll: U.S. Pat. No. 6,546,275: Determination of the arterial input function in dynamic contrast-enhanced MRI
[9] K. Mouridsen, S. Christensen, L Gyldensted, and L. Ostergaard: Automatic selection of arterial input function using cluster analysis. Magn Reson Med, 55(3):524-531, March 2006

We claim:

1. A tomography device for producing magnetic resonance (MR) images of a part of a body of a living organism disposed in a measurement volume of the tomography device, the tomography device comprising:
a first measurement unit, said first measurement unit disposed, structured and dimensioned for acquiring a spatially resolved temporal series of MR images of the part of the body under examination, wherein a temporal series of MR images represents passage of a contrast agent injected into a blood stream of the living organism through an organ located in the part of the body under examination; and
at least one further measurement unit having a local receiver coil, wherein said further measurement unit and said local receiver coil are disposed, structured and dimensioned to measure, close to at least one artery that supplies the part of the body of the living organism disposed in the tomography device, a concentration of the contrast agent with temporal resolution and concurrently with measurement of the temporal series of MR images determined by said first measurement unit.

2. The tomography device of claim 1, wherein said further measurement unit is constituted as a surface coil.

3. The tomography device of claim 1, wherein said further measurement unit is connected to a dedicated data acquisition channel of the tomography device.

4. The tomography device of claim 1, wherein said further measurement unit encloses a smaller measurement volume than said first measurement unit.

5. The tomography device of claim 1, wherein at least two further measurement units are provided.

6. A method for operating the tomography device of claim 1, wherein, in one excitation slice, excitation of a magnetization of the blood and/or contrast agent carried in the at least one artery of the living organism under examination is performed chronologically before and/or after an imaging portion of a measurement sequence of the first measurement unit.

7. The method of claim 6, wherein a read-out operation of the further measurement unit is performed chronologically before and/or after the imaging portion of the measurement sequence of the first measurement unit.

8. The method of claim 7, wherein excitation of the magnetization of the blood and/or contrast agent carried in the artery of the organism under examination is performed before and/or after the imaging portion of the measurement sequence of the first measurement unit, wherein a read-out operation of the further measurement unit is performed during and/or after the imaging portion of the measurement sequence of the first measurement unit.

9. The method of claim 6, wherein the excitation slice is chosen to be sufficiently thick that a change in a signal acquired by the further measurement unit caused by time-variable blood flow in the at least one artery is negligibly small.

10. The method of claim 6, wherein at least two excitation slices are used.

11. The method of claim 6, wherein a contrast between the artery and tissue surrounding the artery is set by means of a flip angle of excitations of the magnetization.

12. The method of claim 7, wherein, subsequent to excitation and read-out operations performed prior to the imaging portion of the measurement sequence, one or more additional excitation and read-out are appended after the imaging portion of the measurement sequence and with variable starting times.

13. The method of claim 6, wherein a contrast agent concentration is determined by isolating a portion of the spectrum caused by the contrast agent out of the measured MR signal of the least one further measurement unit.
